(19) United States
(12) Patent Application Publication
(10) Pub. No.: US 2023/0029043 A1
(43) Pub. Date: Jan. 26, 2023

(54) FLOWRATE CONTROL FOR SELF-PRESSURIZED RESERVOIR OF A DEVICE FOR DELIVERING MEDICATION

(71) Applicant: AITA BIO INC., New York, NY (US)

(72) Inventor: Peter Smeys, San Jose, CA (US)

(21) Appl. No.: 17/783,237

(22) PCT Filed: Dec. 9, 2020
(86) PCT No.: PCT/US20/63971

§ 371 Date: Jun. 7, 2022

Related U.S. Application Data
(60) Provisional application No. 62/946,382, filed on Dec. 10, 2019.
(60) Provisional application No. 62/946,386, filed on Dec. 10, 2019.

(51) Int. Cl. A61M 5/168; A61M 5/142; A61M 5/145; A61M 5/158

(52) U.S. Cl. CPC A61M 5/16881; A61M 5/14244; A61M 5/145; A61M 5/158

(57) ABSTRACT

A device is disclosed that is configured as a fully autonomous and integrated wearable apparatus for diabetes management. The device comprise a self-pressurized reservoir for storing the medication for subsequent delivery to a patient, a needle for delivering the medication to the patient subcutaneously, a first MEMS device configured as a microvalve in a fluid path between the self-pressurized reservoir and needle for controlling flowrate of medication through the needle as the self-pressurized reservoir discharges, a second MEMS device configured as a micropump configured to increase flowrate of the medication in the fluid path to ensure a constant flowrate in the fluid path as the self-pressurized discharges independent of orientation of the device, a flow sensor configured to measure flowrate in the fluid path for controlling microvalve and micropump, and control circuitry connected to the microvalve, micropump and flow sensor for controlling operation of the micropump and microvalve.

### REFERENCE TO RELATED APPLICATIONS

This application claims priority to U.S. provisional application No. 62/946,386, filed Dec. 10, 2019 entitled “Flowrate Control For Self-Pressurized Reservoir of A Device For Delivering Insulin” and U.S. provisional application No. 62/946,382, filed on Dec. 10, 2019 entitled “Device For Delivering Insulin Including Interposer,” which are both incorporated by reference herein.

### FIELD OF THE INVENTION

The present invention relates to flowrate control for a self-pressurized reservoir in a device for delivering medication such as insulin.

### BACKGROUND OF THE INVENTION

Various infusion systems exist that utilize devices for delivering liquid medication or other therapeutic fluid to patients subcutaneously. For patients with diabetes mellitus, for example, conventional infusion systems incorporate various pumps that are used to deliver insulin to a patient. These pumps have the capability of delivering assorted fluid delivery profiles which include specified basal rates and bolus requirements. For example, these pumps include a reservoir to contain the liquid medication along with electromechanical pumping technology to deliver the liquid medication via tubing to a needle that is inserted subcutaneously into the patient. Although such conventional pumps/infusion systems are adequate for their intended purpose, the process of fully emptying the reservoir can be challenging. This can lead to pressure and/or flowrate control for adequate medication delivery.

Therefore, it would be advantageous to provide an improved infusion system over these conventional infusion systems.

### SUMMARY OF THE INVENTION

Flowrate control for a self-pressurized reservoir of a device for delivering insulin or other medication is disclosed.

In accordance with an embodiment of the present disclosure, a device is disclosed that is configured as a fully autonomous and integrated wearable apparatus for diabetes management, the device comprising: a self-pressurized reservoir for storing the medication for subsequent delivery to a patient; a needle for delivering the medication to the patient subcutaneously; a microvalve in a fluid path between the self-pressurized reservoir and needle for controlling flowrate of medication through the needle as the self-pressurized reservoir discharges; a micropump configured to increase flowrate of the medication in the fluid path to ensure a constant flowrate in the fluid path as the pressure decreases as the self-pressurized discharges independent of orientation of the device; a flow sensor configured to measure flowrate in the fluid path for controlling microvalve and micropump; and control circuitry connected to the microvalve, micropump and flow sensor for controlling operation of the micropump and microvalve.

In accordance with another embodiment of this disclosure, a device is disclosed that is configured as a fully autonomous and integrated wearable apparatus for diabetes management, the device comprising: a self-pressurized reservoir for storing the medication for subsequent delivery to a patient; a needle for delivering the medication to the patient subcutaneously; a first MEMS device configured as a microvalve in a fluid path between the self-pressurized reservoir and needle for controlling flowrate of medication through the needle as the self-pressurized reservoir discharges; a second MEMS device configured as a micropump configured to increase flowrate of the medication in the fluid path to ensure a constant flowrate in the fluid path as the self-pressurized discharges independent of orientation of the device; a flow sensor configured to measure flowrate in the fluid path for controlling microvalve and micropump; and control circuitry connected to the microvalve, micropump and flow sensor for controlling operation of the micropump and microvalve.

In accordance with another embodiment of this disclosure, a device is disclosed for delivering fluid to a user, the device comprising: a reservoir for storing the fluid, the reservoir being configured to be self-pressurized; a needle for delivering the fluid to the user subcutaneously; a microvalve communicating with the reservoir for controlling output flowrate of fluid from the reservoir to the needle; a flow sensor configured to measure the flowrate for controlling microvalve and micropump; and a micropump fluidly communicating with reservoir for increasing the flowrate of the fluid to maintain a constant flowrate of the fluid independent of orientation of the device as the reservoir discharges.

### BRIEF DESCRIPTION OF DRAWINGS

FIGS. 1 and 2 depict a block flow diagram of example flowrate control for a self-pressurized reservoir of a device for delivering insulin.

FIGS. 3-5 depict example views of a self-pressurized reservoir of a device for delivering insulin.

FIG. 6 depicts an example graph of flowrate versus time of a self-pressurized reservoir of a device for delivering insulin.

### DETAILED DESCRIPTION OF THE INVENTION

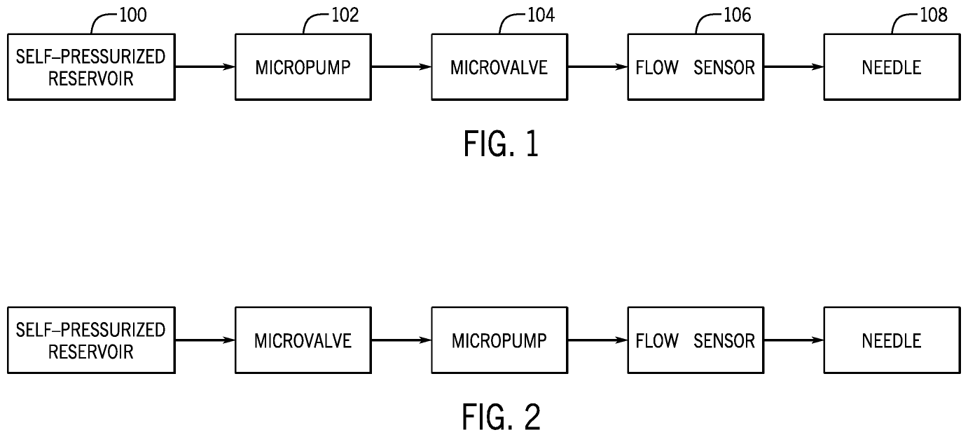

FIGS. 1 and 2 depict a block flow diagram of example flowrate control for a self-pressurized reservoir of a device for delivering insulin to a diabetes patient or user. (The device however may be used for delivering other medication or fluid to a user as known to those skilled in the art.) A self-pressurized reservoir and flowrate control are used as a means to ensure that the reservoir is deflated or emptied in a controlled manner as described in more detail below.

In FIG. 1, the device includes, among other components, a self-pressurized reservoir 100, a micropump 102, microvalve 104, flow sensor 106 and needle 108 (for insulin delivery). The device also includes a continuous glucose monitoring (CGM) or analyte sensor needle (not shown) as known to those skilled in the art. Micropump 102 and microvalve 104 each incorporate MEMS technology (micro-electro-mechanical systems devices) to enable the delivery device to function as a fully autonomous and integrated wearable unit for diabetes management in which continuous glucose monitoring (CGM) or analyte sensing, insulin delivery and control functionality are provided together to ensure insulin is delivered at very precise rates. The device for delivering insulin also includes, among other components, control circuitry connected to micropump 102, microvalve 104 and flow sensor 106. The control circuitry functions (among other functions) to control the operation of the micropump 102 and microvalve 104. (Micropump may be referred to as a small pump or pump and microvalve may be referred to as a small valve or valve.) The device may also include a battery for powering the control circuitry and micropump 102 and microvalve 104 as well as an Interposer configured as an adapter for (1) mounting reservoir 100, micropump 102, microvalve 104, flow sensor 106 and needed 108 control circuitry (integrated circuit—IC) insulin needle and the CGM or analyte sensor needle and for (2) redistributing fluid through channels and electrical signals between those components.

Self-pressurized reservoir 100 is configured to receive and store insulin (or other medication or fluid) for subsequently delivery. Reservoir 100 incorporates a membrane of elastic material such as silicone (or other hyper-elastic material known to those skilled in the art) to enable the membrane to function as a balloon whereby reservoir 100 expands and contracts (i.e., self-pressurized) as insulin is filled and depleted from reservoir 100. The balloon reservoir 100 is thus self-pressurized during filling, by expanding the silicone membrane.

In this example, micropump 102 fluidly communicates with reservoir 104 and microvalve fluidly communicates with micropump 102 and insulin needle 108. As indicated above, micropump 102 and microvalve 104 each incorporate one or more MEMS devices to provide pump and valve functionality as known to those skilled in the art. Micropump 102 and microvalve 104 work together along with pressurized reservoir 100 to enable a controllable flowrate and reservoir discharge independent of the device orientation. Micropump 102 functions as a booster pump to ensure continued fluid pressure as self-pressurized reservoir 100 depletes. That is, microvalve 104 serves to control the flowrate by controlling the hydraulic resistance. As reservoir 100 empties, pressure will drop and flowrate will reduce. Micropump 102 functions as a booster pump in the fluid path to ensure constant flowrate as the pressure drops. In this embodiment, micropump 102 functions as a booster pump as well as traditional pumping functionality for the device for delivering insulin to a diabetes patient or user. However, those skilled in the art know that separate micropump may be employed to provide traditional functional pumping capability while micropump 102 may be used as a booster pump only.

Flow sensor 106 is a sensor for measuring actual flowrate for ultimately controlling microvalve 104 and micropump 102 as known to those skilled in the art. Flow sensor 106 can be a MEMS ultrasound or microthermal flow sensor that is placed in the fluid path. It can be a standalone device, or integrated with the MEMS micropump or microvalve.

In FIG. 2, the same components as in FIG. 1 (i.e., self-pressurized reservoir, microvalve, micropump, flow sensor and needle) are shown and part of a device for delivering insulin (or other medication or fluid). However, the micropump and microvalve are switched in the fluid path, but the device components have the same functionality as those described with respect to FIG. 1. For example, the microvalve and micropump also incorporate one or more MEMS devices.

As indicated above, micropump and microvalve described above incorporate MEMS devices, but those skilled in the art know that other micropumps and microvalves may be used without the MEMS devices.

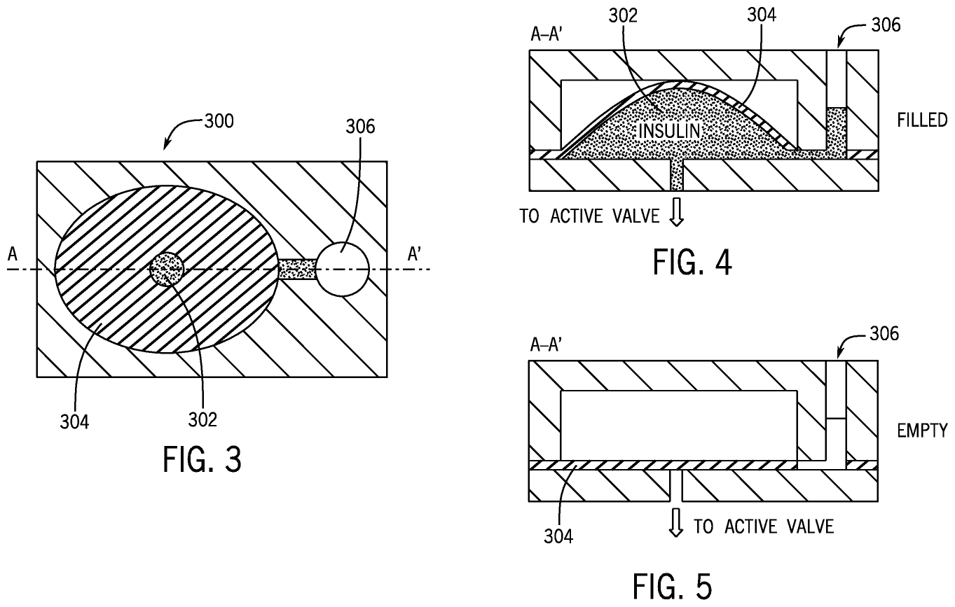

FIGS. 3-5 depict example views of a self-pressurized reservoir of device 300 for delivering insulin (or other medication or fluid). In particular, FIG. 3 depicts a top view of device 300 for delivering insulin including a self-pressurized reservoir 302 defined by silicone membrane 304. FIG. 4 depicts a cross sectional view of the device in FIG. 3 along line A-A′. In this example, reservoir 302 is completely filled with insulin (or alternatively other medications of fluids) and silicone membrane 304 is fully expanded where it contacts the ceiling of the device structure. An active microvalve or (booster) micropump communicates directly with a reservoir 302 via the opening in floor of the reservoir and channel through the device. A silicone stopper 306 is employed to plug and prevent backflow in a channel used to fill the reservoir. FIG. 5 also depicts a cross sectional view of the device in FIG. 3 along line A-A′. However, in this example, reservoir 302 is depleted and silicone membrane 306 is flush against the floor as shown.

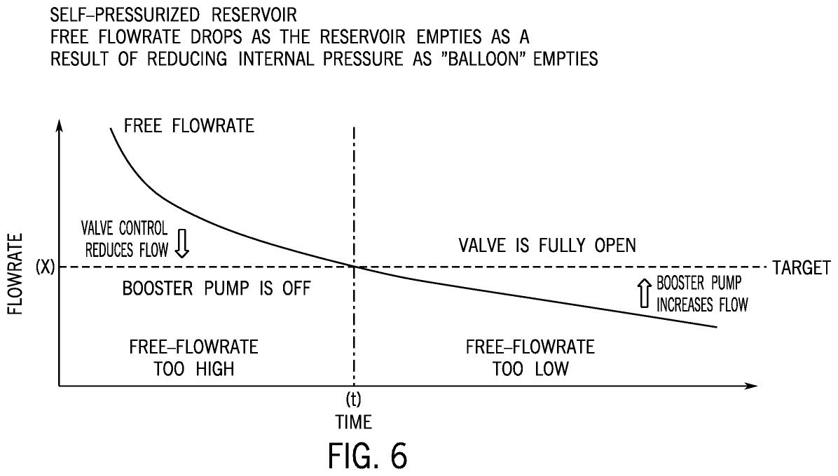

FIG. 6 depicts an example graph of flowrate versus time of a self-pressurized reservoir of a device for delivering insulin (as described hereinabove). (That is, the graph depicts flowrate control for a self-pressurized reservoir.) At the outset, a reservoir is completely filled as depicted in the graph. Free flow rate drops over time as the reservoir discharges insulin (as a result of internal pressure reduction). The microvalve control reduces the flow and the (booster) micropump is off when free-flowrate (or internal reservoir pressure) is too high. As the reservoir empties or discharges and the pressure drops, microvalve is fully opened, and the (booster) micropump is activated to increase flow. The microvalve and micropump act together to balance between flowrate and pressure to ensure constant flowrate. In sum, the micropump is activated at a time in which the flowrate decreases beyond a certain flowrate (X) at time (t) as shown in FIG. 6. That is, the booster pump will increase pressure in the fluid path to thereby increase flowrate to ensure constant flow rate within the device.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. A device configured as a fully autonomous and integrated wearable apparatus for diabetes management, the device comprising:
a self-pressurized reservoir for storing the medication for subsequent delivery to a patient;
a needle for delivering the medication to the patient subcutaneously;
a microvalve in a fluid path between the self-pressurized reservoir and needle for controlling flowrate of medication through the needle as the self-pressurized reservoir discharges;
a micropump configured to increase flowrate of the medication in the fluid path to ensure a constant flowrate in the fluid path as the pressure decreases as the self-pressurized discharges independent of orientation of the device;
a flow sensor configured to measure flowrate in the fluid path for controlling microvalve and micropump; and
control circuitry connected to the microvalve, micropump and flow sensor for controlling operation of the micropump and microvalve.

2. The device of claim 1 wherein the micropump and/or microvalve includes one or more MEMS devices that provide pumping and/or valve functionality.

3. The device of claim 1 wherein the micropump is configured to increase pressure in the fluid path at a time in which flowrate decreases beyond a level.

4. A device configured as a fully autonomous and integrated wearable apparatus for diabetes management, the device comprising:
a self-pressurized reservoir for storing the medication for subsequent delivery to a patient;
a needle for delivering the medication to the patient subcutaneously;
a first MEMS device configured as a microvalve in a fluid path between the self-pressurized reservoir and needle for controlling flowrate of medication through the needle as the self-pressurized reservoir discharges;
a second MEMS device configured as a micropump configured to increase flowrate of the medication in the fluid path to ensure a constant flowrate in the fluid path as the self-pressurized discharges independent of orientation of the device;
a flow sensor configured to measure flowrate in the fluid path for controlling microvalve and micropump; and
control circuitry connected to the microvalve, micropump and flow sensor for controlling operation of the micropump and microvalve.

5. The device of claim 4 wherein the first and second MEMS devices are separate devices.

6. The device of claim 4 wherein the first and second MEMS devices are the same MEMS device with valve and pump functionality.

7. A device for delivering fluid to a user, the device comprising:
a reservoir for storing the fluid, the reservoir being configured to be self-pressurized;
a needle for delivering the fluid to the user subcutaneously;
a microvalve communicating with the reservoir for controlling output flowrate of fluid from the reservoir to the needle;
a flow sensor configured to measure the flowrate for controlling microvalve and micropump; and
a micropump fluidly communicating with reservoir for increasing the flowrate of the fluid to maintain a constant flowrate of the fluid independent of orientation of the device as the reservoir discharges.

8. The device of claim 7 wherein the microvalve includes one or more MEMS devices.

9. The device of claim 7 wherein the micropump includes one or more MEMS devices.
